(19) United States
(12) Patent Application Publication
(10) Pub. No.: US 2007/0027162 A1
(43) Pub. Date: Feb. 1, 2007

(54) Crystalline and amorphous 4-cyano-N-{(2R)-2-[4-(2,3-dihydro-benzo[1,4]dioxin-5-yl)-piperazin -1-yl]-propyl}-N-pyridin-2-yl-benzamide hydrochloride

(75) Inventors: Eric N.C. Browne, Pierrefonds (CA); Michel Bernatchez, Montreal (CA); Mark Lankau, Dollard des Ormeaux (CA); Abdolsamad Tadayon, Kirkland (CA); Anthony F. Hadfield, Ruskin, FL (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/364,902

(22) Filed: Feb. 27, 2006

Related U.S. Application Data
(60) Provisional application No. 60/657,575, filed on Mar. 1, 2005.

(51) Int. Cl. A61K 31/496; C07D 405/14

(52) U.S. Cl. 514/253.11; 544/364

(57) ABSTRACT

The present invention is directed to crystal and amorphous forms of the 5-HT_{1A} receptor antagonist 4-cyano-N-{(2R)-2-[4-(2,3-dihydro-benzo[1,4]dioxin-5-yl)-piperazin-1-yl]-propyl}-N-pyridin-2-yl-benzamide hydrochloride, as well as compositions thereof and methods of using the same.

### CROSS REFERENCE TO RELATED APPLICATIONS

This application claims priority benefit of U.S. Provisional Application Ser. No. 60/657,575, filed Mar. 1, 2005, the entire content of which is incorporated herein by reference.

### FIELD OF THE INVENTION

The present invention is directed to crystal and amorphous forms of the 5-HT_{1A} receptor antagonist 4-cyano-N-{(2R)-2-[4-(2,3-dihydro-benzo[1,4]dioxin-5-yl)-piperazin-1-yl]-propyl}-N-pyridin-2-yl-benzamide hydrochloride, as well as compositions thereof and methods of using the same.

### BACKGROUND OF THE INVENTION

Certain N-aryl-piperazine derivatives act on the central nervous system (CNS) by binding to 5-HT receptors. In pharmacological testing, it has been shown that these derivatives can bind to receptors of the 5-HT_{1A} type and exhibit activity as 5-HT_{1A} antagonists. See, for example, U.S. Pat. Nos. 6,127,357; 6,469,007; and 6,586,436, as well as WO 97/03982, the disclosures of each of which are incorporated herein by reference.

An example N-aryl-piperazine, having 5-HT_{1A} antagonist activity, is 4-cyano-N-{(2R)-2-[4-(2,3-dihydro-benzo[1,4]dioxin-5-yl)-piperazin-1-yl]-propyl}-N-pyridin-2-yl-benzamide, the structure of which (as the HCl salt) is shown below in Formula I. 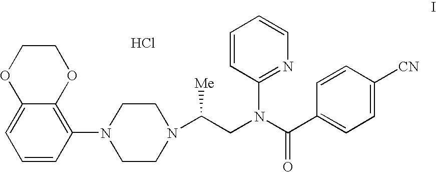
This compound is useful in treating patients suffering from central nervous system (CNS) disorders such as schizophrenia, (and other psychotic disorders such as paranoia and mano-depressive illness), Parkinson's disease and other motor disorders, anxiety (e.g., generalized anxiety disorders, panic attacks, and obsessive compulsive disorders), depression (such as by the potentiation of serotonin reuptake inhibitors and serotonin norepinephrine reuptake inhibitors), Tourette's syndrome, migraine, autism, attention deficit disorders and hyperactivity disorders. This compound can also be useful for the treatment of sleep disorders, social phobias, pain, thermoregulatory disorders, endocrine disorders, urinary incontinence, vasospasm, stroke, eating disorders such as for example obesity, anorexia and bulimia, sexual dysfunction, and the treatment of alcohol, drug and nicotine withdrawal. Additionally, this compound is useful for the treatment of cognitive dysfunction and may be useful for the treatment of cognitive dysfunction associated with mild cognitive impairment (MCI)) Alzheimer's disease and other dementias, including Lewy Body, vascular, and post stroke dementias. Cognitive dysfunction associated with surgical procedures, traumatic brain injury or stroke may also be treated with the compound of Formula I. Further, this compound can be useful for the treatment of diseases in which cognitive dysfunction is a co-morbidity such as, for example, Parkinson's disease, autism, and attention deficit disorders.

The compound of Formula I and related compounds can be prepared according to known procedures such as those described in U.S. Pat. Nos. 6,713,626 and 6,469,007 as well as U.S. App. Ser. No. 60/554,666 and U.S. application Ser. No. 11/082,510 (published as US 2005/0209245A1), each of which is incorporated herein by reference it its entirety. Additionally, pharmaceutical dosage forms and compositions containing the compound of Formula I are described in U.S. App. Ser. No. 60/554,622 and U.S. application Ser. No. 11/082,548 (published as US 2005/0215561A1), which is incorporated herein by reference in its entirety.

Improved drug formulations, showing, for example, better bioavailability or better stability, are consistently sought. There is an ongoing need for new or purer crystalline forms of existing drug molecules. Accordingly, crystalline 4-cyano-N-{(2R)-2-[4-(2,3-dihydro-benzo[1,4]dioxin-5-yl)-piperazin-1-yl]-propyl}-N-pyridin-2-yl-benzamide hydrochloride (I) described herein is directed toward this and other ends.

### SUMMARY OF THE INVENTION

The present invention provides crystal and amorphous forms of 4-cyano-N-{(2R)-2-[4-(2,3-dihydro-benzo[1,4]dioxin-5-yl)-piperazin-1-yl]-propyl}-N-pyridin-2-yl-benzamide hydrochloride (I) characterized according to the X-ray powder diffraction, single crystal X-ray diffraction, differential scanning calorimetry (DSC), therogravimetric analysis (TGA) and other techniques described herein.

The present invention further provides compositions containing the crystal form of the invention.

The present invention further provides processes for preparing crystal forms of the invention.

The present invention further provides methods of antagonizing a 5-HT_{1A} receptor by contacting the receptor with a crystal form of the invention.

The present invention further provides methods of treating CNS disorders and cognitive dysfunction by administering a therapeutically effective amount of a crystal form of the invention to a patient in need of the treatment.

### BRIEF DESCRIPTION OF THE DRAWINGS

FIG. 1 depicts an X-ray powder diffraction (XRPD) pattern characteristic of a crystal form of the invention (designated “Form A”), prepared according to the procedure of Example 1.

FIG. 2 depicts a differential scanning calorimetry (DSC) trace and thermogravimetric analysis (TGA) of Form A prepared according to the procedure of Example 1.

FIG. 3 depicts an ORTEP-type drawing of the compound of Formula 1 crystallized according to the procedures described in Example 7.

FIG. 4 depicts an X-ray powder diffraction (XRPD) pattern characteristic of the amorphous form of 4-cyano-N-{(2R)-2-[4-(2,3-dihydro-benzo[1,4]dioxin-5-yl)-piperazin-1-yl]-propyl}-N-pyridin-2-yl-benzamide hydrochloride.

FIG. 5 depicts a differential scanning calorimetry (DSC) trace of the amorphous form of the invention prepared according to the procedure of Example 8.

FIG. 6 depicts an X-ray powder diffraction (XRPD) pattern characteristic of the amorphous form of 4-cyano-N-{(2R)-2-[4-(2,3-dihydro-benzo[1,4]dioxin-5-yl)-piperazin-1-yl]-propyl}-N-pyridin-2-yl-benzamide free base.

FIG. 7 depicts a differential scanning calorimetry (DSC) trace of the amorphous form of 4-cyano-N-{(2R)-2-[4-(2,3-dihydro-benzo[1,4]dioxin-5-yl)-piperazin-1-yl]-propyl}-N-pyridin-2-yl-benzamide free base.

### DETAILED DESCRIPTION

Crystalline Material and Preparations

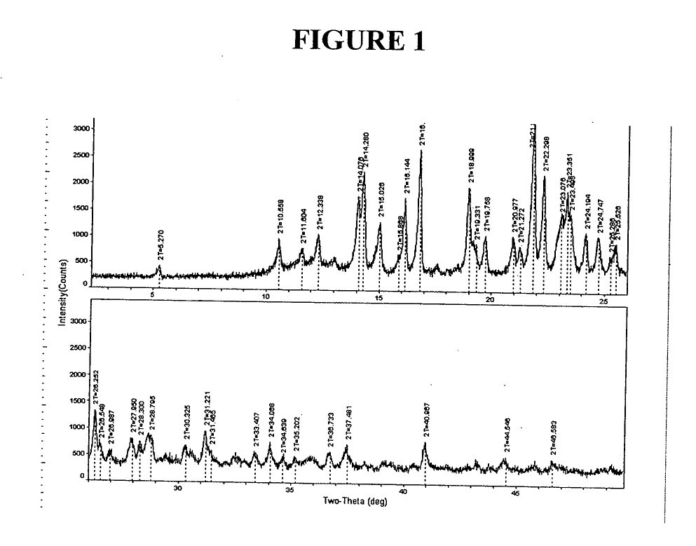

The present invention provides, inter alia, an anhydrous, non-solvated crystal form of 4-cyano-N-{(2R)-2-[4-(2,3-dihydro-benzo[1,4]dioxin-5-yl)-piperazin-1-yl]-propyl}-N-pyridin-2-yl-benzamide hydrochloride (I) having an X-ray powder diffraction pattern substantially as depicted in FIG. 1, designated herein as “Form A.” A list of prominent reflections are provided below in Table 1 along with their corresponding intensities.

TABLE 1

| 2-Theta (°) | Intensity |
|---|---|
| 5.3 | weak |
| 10.6 | moderate |
| 11.6 | weak |
| 12.3 | moderate |
| 14.1 | strong |
| 14.3 | strong |
| 15.0 | strong |
| 16.1 | strong |
| 16.8 | strong |
| 19.0 | strong |
| 19.8 | moderate |
| 21.0 | moderate |
| 21.3 | weak |
| 21.8 | strong |
| 22.3 | strong |
| 23.4 | strong |
| 24.2 | moderate |
| 24.7 | moderate |
| 25.5 | moderate |
| 26.3 | moderate |
| 28.0 | weak |
| 28.8 | weak |
| 31.2 | weak |
| 34.1 | weak |
| 36.7 | weak |
| 37.5 | weak |
| 41.0 | weak |

In some embodiments, the crystal form exhibits an X-ray powder diffraction pattern comprising characteristic peaks, in terms of 20, at about 16.8° and about 21.8°. In some embodiments, the crystal form exhibits an X-ray powder diffraction pattern comprising characteristic peaks, in terms of 20, at about 14.3°, about 16.8°, about 21.8°, and about 22.3°. In some embodiments, the crystal form exhibits an X-ray powder diffraction pattern comprising characteristic peaks, in terms of 20, at about 14.3°, about 16.14°, about 16.8°, about 19.0°, about 21.8°, and about 22.3°. In some embodiments, the crystal form exhibits an X-ray powder diffraction pattern comprising at least 3 characteristic peaks, in terms of 20, selected from about 5.3°, about 10.6°, about 11.6°, about 12.3°, about 14.3°, about 15.0°, about 16.14°, about 16.8°, about 19.0°, about 21.8°, about 22.3°, and about 23.4°. In some embodiments, the crystal form exhibits an X-ray powder diffraction pattern substantially as shown in FIG. 1. As is well known in the art of powder diffraction, the relative intensities of the peaks (reflections) can vary, depending upon the sample preparation technique, the sample mounting procedure and the particular instrument employed. Moreover, instrument variation and other factors can affect the 2-theta values. Therefore, the XRPD peak assignments can vary by plus or minus about 0.2°.

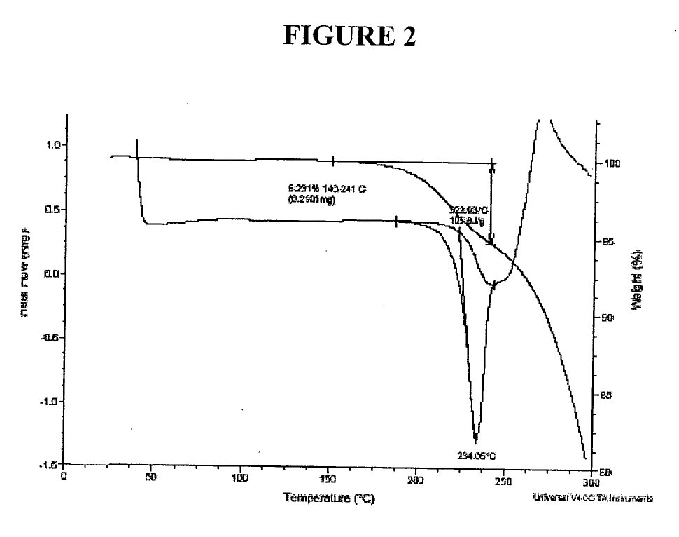

The crystal form having the XPRD pattern of FIG. 1 can also be identified by its characteristic differential scanning (DSC) trace such as shown in FIG. 2. In some embodiments, the DSC exhibits endotherm maximum at about 225 to about 245° C. The endotherm can be characterized as relatively broad. While not wishing to be bound by any particular theory, the breadth of the endotherm is believed to be attributed to decomposition of the sample at these temperatures. In further embodiments, the DSC exhibits an endotherm maximum at about 230 to about 240° C. In further embodiments, the DSC exhibits an endotherm maximum at about 234° C. In yet further embodiments, the crystal form of the invention exhibits a DSC substantially as shown in FIG. 2. For DSC, it is known that the temperatures observed will depend upon the rate of temperature change as well as sample preparation technique and the particular instrument employed. Thus, the values reported herein relating to DSC thermograms can vary by plus or minus about 4° C.

The crystal form having the XPRD pattern of FIG. 1 can also be identified by its characteristic thermogravimetric analysis (TGA) trace such as shown in FIG. 2. In some embodiments, the TGA trace exhibits a feature consistent with about 2.5 to about 7.5% weight loss from about 130 to about 250° C. While not wishing to be bound by theory, the weight loss is believed to be due to loss of HCl as well as decomposition (e.g., loss of a methyl group), as supported by proton NMR data. In further embodiments, the TGA trace exhibits a feature consistent with about 3.5 to about 6.5% weight loss from about 130 to about 250° C. In yet further embodiments, the TGA trace exhibits a feature consistent with about 4.0 to about 6.0% weight loss from about 140 to about 240° C. In some embodiments, the crystal exhibits a TGA trace substantially as shown in FIG. 2. For TGA, it is known that the temperatures observed will depend upon the rate of temperature change as well as sample preparation technique and the particular instrument employed. Thus, the values reported herein relating to TGA thermograms can vary by plus or minus about 4° C.

The crystal form of the invention having, for example, an XRPD pattern according to FIG. 1, can be prepared by any of numerous suitable methods. Fop example, the crystal form can be prepared by precipitating the crystal form from a solution of 4-cyano-N-{(2R)-2-[4-(2,3-dihydro-benzo[1,4]dioxin-5-yl)-piperazin-1-yl]-propyl}-N-pyridin-2-yl-benzamide hydrochloride in a crystallizing solvent. The means of precipitation include any suitable means such as cooling, evaporation, or addition of antisolvent. In some embodiments, the solution is cooled from an elevated temperature of about 50 to about 80° C. to a cooled temperature of about 20 to about −20° C. In some embodiments, the solution is evaporated by, for example, evaporation of a standing solution under ambient condition or evaporation of a solution exposed to a gas stream (e.g., air or inert gas). In some embodiments, addition of antisolvent can be carried out by direct addition of antisolvent to the solution, layered diffusion, or vapor diffusion.

Suitable crystallizing solvents include any solvent in which the compound of Formula I is partially or fully soluble. Example solvents include protic solvents such as water or alcohols (e.g., methanol, ethanol, n-propanol, isopropanol, etc.), other polar solvents such as dimethylsulfoxide, acetonitrile, propionitrile, ethyl acetate, dimethylformamide, dichloromethane, and the like. Other suitable solvents include tetrahydrofuran, toluene, and acetone. In some embodiments, the crystallizing solvent is an alcohol. In further embodiments, the crystallizing solvent is ethanol.

Suitable antisolvents include any solvent in which the compound of Formula I is poorly soluble. Example antisolvents include non-polar or weakly polar solvents such as ethers (diethyl ether, t-butylmethyl ether, etc.) and hydrocarbons (pentane, hexanes, etc.).

The present invention further provides a crystal form of 4-cyano-N-{(2R)-2-[4-(2,3-dihydro-benzo[1,4]dioxin-5-yl)-piperazin-1-yl]-propyl}-N-pyridin-2-yl-benzamide hydrochloride (I) having single crystal X-ray diffraction parameters as shown below in Table 2. Additional parameters, atomic coordinates and other data are provided in Example 7.

TABLE 2

| | |
|---|---|
| Unit cell parameters | a = 8.45 Å |
| | b = 9.30 Å |
| | c = 33.30 Å |
| | α, β, γ = 90° |
| Space group | orthorhombic 2(1)2(1)2(1) (No. 19). |
| Z | 4 |
| Volume | 2621 cubic Å³ |

A crystal form of the invention having one or more of the single crystal parameters recited herein can be prepared according to routine methods. In an example method, the crystal form of the invention can be prepared by precipitating the crystal form from a solution of 4-cyano-N-{(2R)-2-[4-(2,3-dihydro-benzo[1,4]dioxin-5-yl)-piperazin-1-yl]-propyl}-N-pyridin-2-yl-benzamide hydrochloride in a crystallizing solvent by addition of antisolvent. The addition of antisolvent can be carried out by any suitable method such as by direct addition or by vapor diffusion. Suitable antisolvents include ethers (such as diethyl ether or t-butylmethyl ether) and hydrocarbons (such as pentane, hexanes, etc.), and other low boiling solvents. In some embodiments, the antisolvent contains hexanes. The crystallizing solvent can be any of the crystallizing solvent recited hereinbefore. In some embodiments, the crystallizing solvent contains an alcohol. In some embodiments, the crystallizing solvent is ethanol.

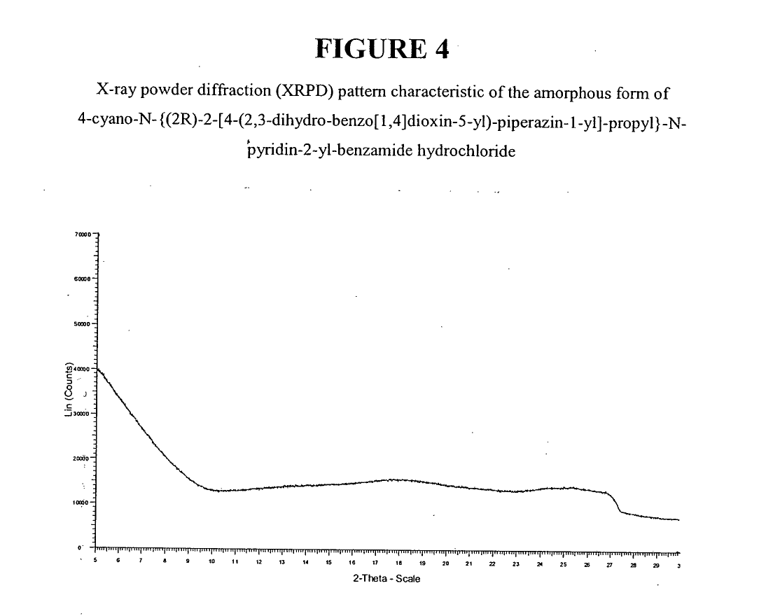

The present invention further provides an amorphous form of 4-cyano-N-{(2R)-2-[4-(2,3-dihydro-benzo[1,4]dioxin-5-yl)-piperazin-1-yl]-propyl}-N-pyridin-2-yl-benzamide hydrochloride (I). As can be seen from FIG. 4, the X-ray powder diffraction pattern of the amorphous form is substantially devoid of any prominent peaks (reflections).

Compositions, Formulations, and Dosage Forms

The present invention further provides a composition containing a crystal form of the invention. In some embodiments, at least about 50%, at least about 60%, at least about 70%, at least about 80%, at least about 90%, at least about 95%, at least about 97%, at least about 98%, or at least about 99% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in the composition is present as the crystal form. In some of each such embodiments, less than about 10%, less than about 5%, less than about 3%, less than about 2%, less than about 1%, less than about 0.5%, or less than about 0.1% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in the composition is present as the amorphous form. In further embodiments, the composition is substantially free of the amorphous form of the hydrochloride. In further embodiments, the composition is a pharmaceutical composition containing a crystal form of the invention and a pharmaceutically acceptable carrier.

The present invention further provides a composition containing the amorphous form of the invention. In some embodiments, at least about 50%, at least about 60%, at least about 70%, at least about 80%, at least about 90%, at least about 95%, at least about 97%, at least about 98%, or at least about 99% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in the composition is present as the amorphous form. In further embodiments, the composition is a pharmaceutical composition containing the amorphous form of the invention and a pharmaceutically acceptable carrier.

The crystal and amorphous forms of the present invention can be administered orally or parentally, neat or in combination or association with conventional pharmaceutical carriers. Applicable solid carriers can include one or more substances which may also act as flavoring agents, lubricants, solubilizers, suspending agents, fillers, glidants, compression aids, binders, tablet-disintegrating agents or encapsulating materials. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets may contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins. Liquid carriers may be used in preparing solutions, suspensions, emulsions, syrups and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g., cellulose derivatives, preferably sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. Liquid pharmaceutical compositions which are sterile solutions or suspensions can be utilized by, for example, intramuscular, intraperitoneal or subcutaneous injection. Sterile solutions can also be administered intravenously. Oral administration may be either in liquid or solid composition form. Preferably, the pharmaceutical compositions containing the present crystal forms are in unit dosage form, e.g., as tablets or capsules. In such form, the composition is sub-divided in unit dosages containing appropriate quantities of the active ingredients. The unit dosage forms can be packaged compositions, for example, packaged powders, vials, ampoules, prefilled syringes or sachets containing liquids. Alternatively, the unit dosage form can be, for example, a capsule or tablet itself, or it can be the appropriate number of any such compositions in package form. The therapeutically effective dosage to be used may be varied or adjusted by the physician and generally ranges from 0.5 mg to 750 mg, according to the specific condition(s) being treated and the size, age and response pattern of the patient.

Further example dosage forms and compositions are described in U.S. App. Ser. No. 60/554,622 and U.S. application Ser. No. 11/082,548 (published as U.S. 2005/0215561A1), which is incorporated herein by reference in its entirety.

Methods of Use

As antagonists of the 5-HT_{1A} receptor, the crystal forms of the invention can useful in inhibiting the activity of the receptor. The inhibiting can be carried out, for example, by contacting the crystal form with the receptor in vitro, in vivo, or ex vivo. Accordingly, the crystal or amorphous forms of the present invention can be used to treat a subject (e.g., patient, individual, etc.) suffering from CNS disorders such as schizophrenia, (and other psychotic disorders such as paranoia and mano-depressive illness), Parkinson's disease and other motor disorders, anxiety (e.g. generalized anxiety disorders, panic attacks, and obsessive compulsive disorders), depression (such as by the potentiation of serotonin reuptake inhibitors and serotonin norepinephrine reuptake inhibitors), Tourette's syndrome, migraine, autism, attention deficit disorders and hyperactivity disorders. Crystal and amorphous forms of the present invention can also be useful for the treatment of sleep disorders, social phobias, pain, thermoregulatory disorders, endocrine disorders, urinary incontinence, vasospasm, stroke, eating disorders such as for example obesity, anorexia and bulimia, sexual dysfunction, and the treatment of alcohol, drug and nicotine withdrawal.

Crystal and amorphous forms of the present invention are also useful for the treatment of cognitive dysfunction. Thus, crystal forms of the present invention may be useful for the treatment of cognitive dysfunction associated with mild cognitive impairment (MCI)) Alzheimer's disease and other dementias including Lewy Body, vascular, and post stroke dementias. Cognitive dysfunction associated with surgical procedures, traumatic brain injury or stroke may also be treated in accordance with the present invention. Further, crystal or amorphous forms of the present invention may be useful for the treatment of diseases in which cognitive dysfunction is a co-morbidity such as, for example, Parkinson's disease, autism and attention deficit disorders.

Treatment of a patient can be carried out by administering a therapeutically effective amount of a crystal or amorphous form of the compound of Formula I to a patient in need of treatment. Suitable patients are, for example, mammals, especially humans, suffering from or likely to suffer from any of the CNS disorders or cognitive dysfunctions listed above, or other 5-HT_{1A} receptor-associated disease.

As used herein, the term “individual” or “patient” or “subject,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or symptomatology of the disease, condition or disorder (i.e., arresting further development of the pathology and/or symptomatology) such as stabilizing viral load in the case of a viral infection; and

(3) ameliorating the disease; for example, ameliorating a disease, condition or disorder in an individual that is experiencing or displaying the pathology or symptomatology of the disease, condition or disorder (i.e., reversing the pathology and/or symptomatology) such as lowering viral load in the case of a viral infection.

One or more additional pharmaceutical agents can be used in combination with the crystal forms of the present invention for treatment of 5-HT_{1A}-associated diseases, disorders or conditions. The agents can be combined with the present compounds in a single dosage form, or the agents can be administered simultaneously or sequentially as separate dosage forms.

In some embodiments of each of the crystal forms described herein, the crystal forms are provided in a form that is substantially free of hydrocarbon solvents, such as hexane and heptane. Such solvents have been used in the later stages of preparation of 4-cyano-N-{(2R)-2-[4-(2,3-dihydro-benzo[1,4]dioxin-5-yl)-piperazin-1-yl]-propyl}-N-pyridin-2-yl-benzamide hydrochloride, for example in the procedures described in U.S. Pat. Nos. 6,713,626 and 6,469,007, and U.S. App. Ser. No. 60/554,666, described supra. In such cases, it is desirable to further purify the preparations of the compound to remove traces of such solvents that may remain in the preparation. This can be accomplished by any of a variety of standard techniques, including one or more recrystallizations from a more pharmacologically acceptable solvent, such as ethanol, or by additional drying or chromatography procedures, or other procedures used for the removal of impurities from pharmaceuticals.

As used herein, the term “substantially free” as applied to a chemical species, is intended to mean that the indicated species is present in less than about 0.01% by weight, relative to the total weight of the sample.

In order that the invention disclosed herein may be more efficiently understood, examples are provided below. It should be understood that these examples are for illustrative purposes only and are not to be construed as limiting the invention in any manner.

### EXAMPLES

### Example 1

### PREPARATION OF CRYSTALLINE 4-CYANO-N-{(2R)-2-[4-(2,3-DIHYDRO-BENZO[1,4]DIOXIN-5-YL)-PIPERAZIN-1-YL]-PROPYL}-N-PYRIDIN-2-YL-BENZAMIDE HYDROCHLORIDE (I)

4-Cyano-N-{(2R)-2-[4-(2,3-dihydro-benzo[1,4]dioxin-5-yl)-piperazin-1-yl]-propyl}-N-pyridin-2-yl-benzamide hydrochloride (15 Kg) was combined with 105 Kg of ethanol 2B (standard commercial form of anhydrous ethanol that typically consists of 99.5% ethanol and 0.5% toluene (by weight)) and the resulting mixture was heated to reflux (approx. 78° C.). Once dissolution was complete, the mixture was cooled to 60-65° C. and clarified by filtration through a 0.2 micron cartridge filter. Additional hot (60-70° C.) ethanol 2B, 30 Kg, was used to rinse the vessel and filter cartridge. The combined filtrates were concentrated to a volume of 86 L by vacuum distillation (maximum pot temperature=40° C.). The reduced solution was then heated to reflux and held for 10 minutes. The solution was then cooled to 15-25° C. over 1 hour followed by stirring for a minimum of 2 hours. The mixture was then cooled further to −15 to −5° C. over 1 hour followed by stirring for a minimum of 2 hours. The crystallized product was filtered and then washed with two 15 Kg portions of ethanol 2B. The material thus obtained was then dried at 60° C. under vacuum. Yield: 11.3 Kg.

Micronization: The crystalline material obtained above was first comilled using a 0.094″ screen at 1200 to 1400 RPM. The resulting material was then micronized using 35 PSI nitrogen at a feed rate of 50 to 80 grams/minute with 80 CFM jets using a T-15 trost mill micronizer to yield a fine crystalline powder.

### Example 2

### Solubility Determination

The solubility of the compound of Formula I in a variety of solvents was measured according to routine methods at 23° C. and 50° C. Results are presented below in Table A.

TABLE A

| | Solubility at 23° C. | Solubility at 50° C. |
|---|---|---|
| Solvent | mg/mL | mg/mL |
| MeOH | 378 | 989 |
| DMSO | 246 | 337 |
| Water | 49 | >100 |
| EtOH | 19 | 40 |
| CH₃CN | 18 | 37 |
| Ethyl acetate | 16 | 24 |
| Toluene | 8 | 14 |
| Acetone | 6 | 9 |
| THF | 4 | 5 |
| 2-Propanol | 2 | 6 |
| Heptane | 1 | 1 |
| t-Butylmethyl ether (t-BME) | 0 | 1 |

### Example 3

### Thermogravimetric Analysis (TGA) and Differential Scanning Calorimetry (DSC) Analysis

The crystal form of the invention, prepared according to Example 1, was analyzed by TGA and DSC by heating a 2-10 mg sample in a platinum cup under nitrogen flow from 25 to 300° C. at a linear scan rate of 10°/min using a Q600 SDT DSC/TGA instrument (TA Instruments). A representative spectrum is provided in FIG. 2. DSC data revealed a broad endotherm at about 234° C., and TGA data showed a weight loss in the range of about 140-240° C. of about 5.2%. The weight loss is believed to be due to loss of HCl and a methyl group as suggested by proton NMR spectra of samples after heating to 240° C.

### Example 4

### Polymorph Screen by Reslurry

The crystal form of the invention remained stable upon slurrying in a variety of different solvents at 23° C. and 50° C. Thermogravimetric analysis (TGA) and differential scanning calorimetry (DSC) data of products of various re-slurries are compared in Tables B and C below together with TGA/DSC data from the crystal form of the invention prepared according to Example 1. TGA/DSC data was obtained as described in Example 3. While DSC endotherms and TGA weight losses slightly differ between experiments, the variation is expected for HCl loss and decomposition of the sample. Powder X-ray diffraction data (see below Examples) of samples from each of the slurries were consistent with the diffraction pattern of FIG. 1 and Table 1.

TABLE B
23° C.

| Slurry Solvent | DSC data | TGA data |
|---|---|---|
| no slurry/Example 1 | Broad endotherm T apex 234.0° C. | 5.2% weight loss 140-241° C. |
| Methanol | Broad endotherm T apex 236.4° C. | 4.5% weight loss 165-243° C. |
| Ethanol | Broad endotherm T apex 237.0° C. | 4.5% weight loss 175-241° C. |
| 2-Propanol | Broad endotherm T apex 238.8° C. | 4.6% weight loss 165-241° C. |
| Acetone | Broad endotherm T apex 239.7° C. | 4.3% weight loss 165-241° C. |
| CH3CN | Broad endotherm T apex 240.6° C. | 5.0% weight loss 165-245° C. |
| Ethyl acetate | Broad endotherm T apex 240.8° C. | 5.1% weight loss 165-243° C. |
| THF | Broad endotherm T apex 239.9° C. | 4.7% weight loss 165-241° C. |
| Toluene | Broad endotherm T apex 238.3° C. | 6.0% weight loss 165-240° C. |
| t-BME | Broad endotherm T apex 237.6° C. | 6.1% weight loss 165-246° C. |
| DMSO | Broad endotherm T apex 239.8° C. | 3.7% weight loss 165-241° C. |
| heptane | Broad endotherm T apex 235.8° C. | 4.4% weight loss 165-243° C. |
| Water | Broad endotherm T apex 233.7° C. | 4.8% weight loss 165-245° C. |

TABLE C
50° C.

| Slurry Solvent | DSC data | TGA data |
|---|---|---|
| Methanol | Broad endotherm T | 5.1% weight loss 165-234° C. |
| | apex 233.8° C. | |
| Ethanol | Broad endotherm T | 6.3% weight loss 165-241° C. |
| | apex 237.25° C. | |
| 2-Propanol | Broad endotherm T | 4.5% weight loss 165-243° C. |
| | apex 239.3° C. | |
| Acetone | Broad endotherm T | 5.4% weight loss 165-243° C. |
| | apex 238.7° C. | |
| CH₃CN | Broad endotherm T | 5.2% weight loss 165-242° C. |
| | apex 238.9° C. | |
| Ethyl acetate | Broad endotherm T | 4.4% weight loss 165-244° C. |
| | apex 241.3° C. | |
| THF | Broad endotherm T | 4.8% weight loss 165-240° C. |
| | apex 240.4° C. | |
| Toluene | Broad endotherm T | 5.4% weight loss 165-244° C. |
| | apex 237.7° C. | |
| DMSO | Broad endotherm T | 3.0% weight loss 165-242° C. |
| | apex 237.5° C. | |

### Example 5

### Polymorph Screen by Cooling, Evaporation, and Antisolvent Techniques

The crystal form of the invention was obtained by crystallization from various solutions. Differential scanning calorimetry (DSC) data of products of various crystallizations are compared in Tables D, E and F below. Table D contains data for crystalline material obtained by cooling solutions of the compound of Formula I in the solvents listed. For example, a saturated solution of the compound of Formula I in the specified solvent at about 50° C. was cooled to about 20-25° C. and the resulting crystalline material analyzed. Table E contains data for crystalline material obtained by evaporation of solutions of the compound of Formula I using solvents listed. For example, evaporation was carried out by gradually warming a saturated solution of the compound of Formula I or by leaving a saturated solution of the compound of Formula I in a vial (covered by A1 foil or perforated paraffin) exposed to air for enough time to generate crystalline solid. Table E contains data for crystalline material obtained by antisolvent methods (e.g., adding antisolvent to a saturated solution of the compound of Formula I or adding a saturated solution of compound of Formula I to antisolvent) using the solvents listed and t-BMS as antisolvent. Use of water or ethanol in the evaporation experiments resulted only in oils. DSC data was obtained as described in Example 3. And, as with the reslurry experiments of Example 4, the DSC endotherms slightly differ between experiments, and are accounted for by HCl loss and decomposition of the sample. Powder X-ray diffraction data (see below Examples) of samples from each of the cooling, evaporation, and antisolvent experiments were consistent with FIG. 1 and Table 1.

TABLE D
Cooling crystallization

| Solvent | DSC data |
|---|---|
| Methanol | Broad endotherm T apex 234.2° C. |
| Ethanol | Broad endotherm T apex 235.0° C. |
| CH₃CN | Broad endotherm T apex 236.4° C. |
| DMSO | Broad endotherm T apex 236.8° C. |

TABLE E
Evaporation crystallization

| Solvent | DSC data |
|---|---|
| Methanol | Broad endotherm T apex 235.7° C. |
| CH₃CN | Broad endotherm T apex 235.5° C. |
| Ethyl acetate | Broad endotherm T apex 234.6° C. |
| DMSO | Broad endotherm T apex 231.2° C. |

TABLE F
Antisolvent crystallization

| Solvent | DSC data |
|---|---|
| Methanol | Broad endotherm T apex 227.5° C. |
| CH₃CN | Broad endotherm T apex 238.0° C. |
| Ethyl acetate | Broad endotherm T apex 239.5° C. |
| DMSO | Broad endotherm T apex 231.7° C. |

### Example 6

### X-Ray Powder Diffraction Data

X-Ray powder diffraction (XRPD) data was collected on a sample of the compound of Formula I prepared according to Example 1 using a Rigaku Miniflex Diffractioin System (Rigaku MSC, Inc.). Powder samples were deposited on a zero-background polished silicon sample holder. A normal focus copper X-ray tube at 0.45 kW equipped with a Ni K-beta filter scanning at 1.0 degree/min from 3.00 to 40.00 degree 2-theta was used as the X-ray source. Data processing was carried out using Jade 6.0 software. Similarly, XRPD data was acquired for the samples obtained from the polymorph screens of Examples 4 and 5. One diffraction pattern was consistently observed and is provided in FIG. 1. A list of reflections is provided above in Table 1.

### Example 7

### Single Crystal X-Ray Data

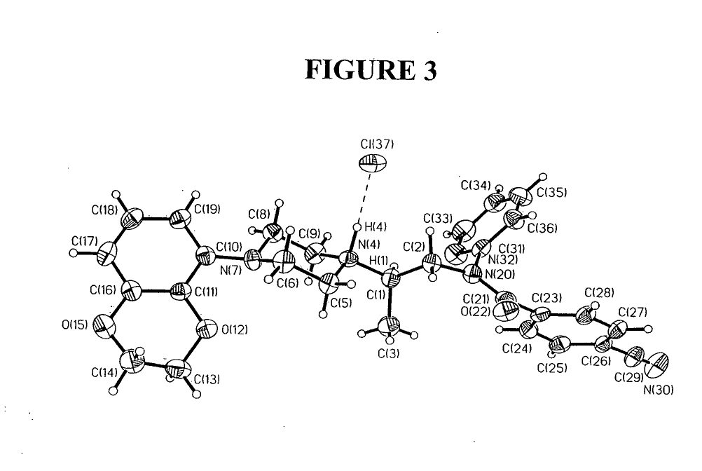

The X-ray structure was determined for the compound of Formula I. The ORTEP drawing is provided in FIG. 3 and coordinates, distances, angles and collection data are provided below in Tables G, H, I, J, K and L.

Single crystals (colorless needles) of a compound of Formula I were obtained from EtOH/hexanes. A single needle, cut to 0.05 mm×0.10 mm×0.22 mm in size, was mounted on a glass fiber with silicone grease and transferred to a Nonius Kappa CCD diffractometer equipped with an MSC X-stream cryosystem and molybdenum K-α radiation (λ=0.71073 Å). Six hundred frames of data were collected at 200(2) K with an omega oscillation range of 0.5 degree/frame, and an exposure time of 240 seconds/degree. A total of 10,607 reflections (θ maximum=22.50°) were indexed, integrated and corrected for Lorentz and polarization effects using DENZO-SMN and SCALEPACK. A Gaussian Face-Indexed absorption correction was then applied using SHELXTL to give 3416 unique reflections (Rᵢₙₜ=0.0844) of which 2933 had I>2σ(I). The minimum and maximum transmission factors were 0.98135 and 0.99183, respectively. Post-refinement of the unit cell parameters gave a=8.4682(4) Å, b=9.2948(3) Å, c=33.2986(15) Å, alpha=beta=gamma=90°, and V=2620.9(2) cubic Å. Axial photographs and systematic absences were consistent with the compound having crystallized in the orthorhombic space group P2(1)2(1)2(1) (No. 19). The observed mean |E*E-1| value was 0.777 (versus the expectation values of 0.968 and 0.736 for centric and noncentric data, respectively).

The structure was solved by direct methods and refined by full-matrix least-squares on F² using SHELXTL The coordinates and anisotropic displacement coefficients for the nonhydrogen atoms were free to vary. The coordinates for the piperazinium hydrogen H(4) were also refined, while those for the remaining hydrogens were allowed to ride on their respective carbons. The hydrogen atoms were assigned isotropic displacement coefficients U(H)=1.2U(C), 1.5U(C_{methyl}) or 1.5U(N), and the weighting scheme employed was w=1/[\s²(Fo²)+(0.0209P)²+0.5003P] where P=(Fo²+2Fc²)/3. The refinement converged to R(F)=0.0518, wR(F²)=0.0944, and S=1.118 for 2933 reflections with I>2sigma(I), and R(F)=0.0665, wR(F²)=0.1027, and S=1.118 for 3416 unique reflections and 338 parameters. The maximum |delta/sigma| in the final cycle of least-squares was less than 0.001, and the residual peaks on the final difference-Fourier map ranged from −0.178 to 0.234 electrons/cubic Angstroms. Scattering factors were taken from the International Tables for Crystallography, Volume C.

The Flack parameter refined to −0.11(10) [versus the expectation values of 0 for the correct hand and 1 for the wrong hand] indicating that the hand of the molecule can be unequivocally assigned as (1R).

For comparison, a refinement of the inverted molecule having the wrong absolute structure, i.e., (1S), gave R(F)=0.0526, wR(F²)=0.0972, and S=1.114 for 2933 reflections with I>2σ(I), and R(F)=0.0673, wR(F²)=0.1057, and S=1.113 for 3416 unique reflections and 338 parameters. The Flack parameter based on the wrong absolute structure was 1.10(10).

TABLE G
Single Crystal Data and Structure Refinement

| Name: | 4-Cyano-N-{(2R)-2-[4-(2,3-dihydro- | |
|---|---|---|
| | benzo[1,4]dioxin-5-yl)-piperazin-1- | |
| | yl]-propyl}-N-pyridin-2- | |
| | yl-benzamide Hydrochloride | |
| Empirical formula | C₂₈H₃₀ClN₅O₃ | |
| Formula weight | 520.02 | |
| Temperature | 200(2) K | |
| Wavelength | 0.71073 Å | |
| Crystal system, space group | Orthorhombic, 2(1)2(1)2(1) (No. 19) | |
| Unit cell dimensions | a = 8.4682(4) Å | alpha = 90 deg. |
| | b = 9.2948(3) Å | beta = 90 deg. |
| | c = 33.2986(15) Å | gamma = 90 deg. |

| | |
|---|---|
| Volume | 2620.9(2) Å³ |
| Z, Calculated density | 4, 1.318 Mg/m³ |
| Absorption coefficient | 0.185 mm⁻¹ |
| F(000) | 1096 |
| Crystal size | 0.22 × 0.10 × 0.05 mm |
| Theta range for data collection | 1.22 to 22.50 deg. |
| Limiting indices | −9 <= h <= 9, −9 <= k <= 10, |
| | −29 <= 1 <= 35 |
| Reflections collected/unique | 10607/3416 [R(int) = 0.0844] |
| Completeness to theta = 22.50 | 100.0% |
| Absorption correction | Gaussian |
| Max. and min. transmission | 0.99183 and 0.98135 |
| Refinement method | Full-matrix least-squares on F² |
| Data/restraints/parameters | 3416/0/338 |
| Goodness-of-fit on F\hat{ }2 | 1.118 |
| Final R indices [I > 2sigma(I)] | R1 = 0.0518, wR² = 0.0944 |
| R indices (all data) | R1 = 0.0665, wR² = 0.1027 |
| Absolute structure parameter | −0.11(10) |
| Largest diff. peak and hole | 0.234 and −0.178 e.A⁻³ |

TABLE H
Atomic coordinates (×10⁴) and equivalent isotropic displacement
parameters (Å² × 10³). U (eq) is defined as one third of the trace of the
orthogonalized Uij tensor.

| U (eq) | x | y | z |
|---|---|---|---|
| C (1) | 8258 (4) | 4692 (4) | 2633 (1) |
| 34 (1) | | | |
| C (2) | 7791 (4) | 3693 (4) | 2975 (1) |
| 34 (1) | | | |
| C (3) | 9883 (5) | 4351 (5) | 2465 (1) |
| 56 (1) | | | |
| N (4) | 6971 (3) | 4691 (3) | 2316 (1) |
| 31 (1) | | | |
| C (5) | 7069 (5) | 3446 (4) | 2027 (1) |
| 37 (1) | | | |
| C (6) | 5757 (5) | 3552 (4) | 1716 (1) |
| 40 (1) | | | |
| N (7) | 5860 (3) | 4934 (3) | 1507 (1) |
| 33 (1) | | | |
| C (8) | 5610 (5) | 6114 (4) | 1789 (1) |
| 36 (1) | | | |
| C (9) | 6918 (5) | 6098 (4) | 2094 (1) |
| 38 (1) | | | |
| C (10) | 5156 (4) | 5038 (4) | 1126 (1) |
| 33 (1) | | | |
| C (11) | 5794 (4) | 4247 (4) | 806 (1) |
| 32 (1) | | | |
| O (12) | 7076 (3) | 3355 (3) | 893 (1) |
| 41 (1) | | | |
| C (13) | 7949 (5) | 2988 (4) | 543 (1) |
| 46 (1) | | | |
| C (14) | 6888 (5) | 2512 (4) | 214 (1) |
| 49 (1) | | | |
| O (15) | 5830 (3) | 3646 (3) | 98 (1) |
| 48 (1) | | | |
| C (16) | 5214 (5) | 4387 (4) | 420 (1) |
| 34 (1) | | | |
| C (17) | 3987 (5) | 5351 (4) | 339 (1) |
| 42 (1) | | | |
| C (18) | 3353 (5) | 6136 (4) | 653 (1) |
| 41 (1) | | | |
| C (19) | 3893 (4) | 5976 (4) | 1040 (1) |
| 36 (1) | | | |
| N (20) | 8844 (4) | 3913 (3) | 3323 (1) |
| 34 (1) | | | |
| C (21) | 9699 (5) | 2764 (5) | 3474 (1) |
| 37 (1) | | | |
| O (22) | 9484 (3) | 1522 (3) | 3353 (1) |
| 43 (1) | | | |
| C (23) | 10938 (4) | 3101 (4) | 3778 (1) |
| 31 (1) | | | |
| C (24) | 11977 (5) | 4239 (4) | 3719 (1) |
| 38 (1) | | | |
| C (25) | 13174 (5) | 4475 (4) | 3994 (1) |
| 39 (1) | | | |
| C (26) | 13314 (4) | 3602 (4) | 4330 (1) |
| 34 (1) | | | |
| C (27) | 12275 (5) | 2448 (4) | 4387 (1) |
| 39 (1) | | | |
| C (28) | 11121 (5) | 2203 (4) | 4105 (1) |
| 36 (1) | | | |
| C (29) | 14509 (6) | 3911 (4) | 4631 (2) |
| 45 (1) | | | |
| N (30) | 15418 (5) | 4219 (4) | 4866 (1) |
| 62 (1) | | | |
| C (31) | 8599 (4) | 5177 (4) | 3559 (1) |
| 35 (1) | | | |
| N (32) | 9042 (4) | 6432 (4) | 3398 (1) |
| 46 (1) | | | |
| C (33) | 8771 (5) | 7622 (4) | 3617 (2) |
| 50 (1) | | | |
| C (34) | 8102 (5) | 7605 (5) | 3993 (1) |
| 46 (1) | | | |
| C (35) | 7661 (5) | 6290 (5) | 4148 (1) |
| 51 (1) | | | |
| C (36) | 7901 (4) | 5054 (4) | 3928 (1) |
| 41 (1) | | | |
| Cl (37) | 3931 (1) | 4848 (1) | 2790 (1) |
| 48 (1) | | | |

TABLE I
Bond lengths [Å] and angles [deg].

| | |
|---|---|
| C(1)—N(4) | 1.515(4) |
| C(1)—C(3) | 1.519(5) |
| C(1)—C(2) | 1.522(5) |
| C(2)—N(20) | 1.477(5) |
| N(4)—C(9) | 1.504(4) |
| N(4)—C(5) | 1.509(4) |
| N(4)—H(4) | 1.04(4) |
| C(5)—C(6) | 1.521(5) |
| C(6)—N(7) | 1.465(4) |
| N(7)—C(10) | 1.403(5) |
| N(7)—C(8) | 1.460(4) |
| C(8)—C(9) | 1.504(5) |
| C(10)—C(11) | 1.403(5) |
| C(10)—C(19) | 1.410(5) |
| C(11)—C(16) | 1.384(5) |
| C(11)—O(12) | 1.397(4) |
| O(12)—C(13) | 1.424(4) |
| C(13)—C(14) | 1.484(6) |
| C(14)—O(15) | 1.436(5) |
| O(15)—C(16) | 1.377(4) |
| C(16)—C(17) | 1.398(5) |
| C(17)—C(18) | 1.383(5) |
| C(18)—C(19) | 1.378(5) |
| N(20)—C(21) | 1.385(5) |
| N(20)—C(31) | 1.429(5) |
| C(21)—O(22) | 1.236(4) |
| C(21)—C(23) | 1.492(5) |
| C(23)—C(28) | 1.380(5) |
| C(23)—C(24) | 1.390(5) |
| C(24)—C(25) | 1.384(5) |
| C(25)—C(26) | 1.388(5) |
| C(26)—C(27) | 1.400(5) |
| C(26)—C(29) | 1.454(6) |
| C(27)—C(28) | 1.376(5) |
| C(29)—N(30) | 1.134(5) |
| C(31)—N(32) | 1.338(5) |
| C(31)—C(36) | 1.367(5) |
| N(32)—C(33) | 1.344(5) |
| C(33)—C(34) | 1.375(6) |
| C(34)—C(35) | 1.377(6) |
| C(35)—C(36) | 1.377(5) |
| N(4)—C(1)—C(3) | 113.3(3) |
| N(4)—C(1)—C(2) | 109.5(3) |
| C(3)—C(1)—C(2) | 112.5(3) |
| N(20)—C(2)—C(1) | 110.2(3) |
| C(9)—N(4)—C(5) | 110.7(3) |
| C(9)—N(4)—C(1) | 111.3(3) |
| C(5)—N(4)—C(1) | 113.9(3) |
| N(4)—C(5)—C(6) | 110.1(3) |
| N(7)—C(6)—C(5) | 109.7(3) |
| C(10)—N(7)—C(8) | 117.8(3) |
| C(10)—N(7)—C(6) | 117.7(3) |
| C(8)—N(7)—C(6) | 110.1(3) |
| N(7)—C(8)—C(9) | 108.7(3) |
| N(4)—C(9)—C(8) | 111.4(3) |
| N(7)—C(10)—C(11) | 119.1(3) |
| N(7)—C(10)—C(19) | 123.3(4) |
| C(11)—C(10)—C(19) | 117.5(4) |
| C(16)—C(11)—O(12) | 121.6(4) |
| C(16)—C(11)—C(10) | 121.4(4) |
| O(12)—C(11)—C(10) | 116.9(4) |
| C(11)—O(12)—C(13) | 112.1(3) |
| O(12)—C(13)—C(14) | 111.3(4) |
| O(15)—C(14)—C(13) | 111.0(3) |
| C(16)—O(15)—C(14) | 113.2(3) |
| O(15)—C(16)—C(11) | 122.9(4) |
| O(15)—C(16)—C(17) | 116.9(4) |
| C(11)—C(16)—C(17) | 120.2(4) |
| C(18)—C(17)—C(16) | 118.8(4) |
| C(19)—C(18)—C(17) | 121.5(4) |
| C(18)—C(19)—C(10) | 120.6(4) |
| C(21)—N(20)—C(31) | 120.7(3) |
| C(21)—N(20)—C(2) | 119.5(3) |
| C(31)—N(20)—C(2) | 117.3(3) |
| O(22)—C(21)—N(20) | 121.8(4) |
| O(22)—C(21)—C(23) | 121.3(4) |
| N(20)—C(21)—C(23) | 116.9(4) |
| C(28)—C(23)—C(24) | 120.0(4) |
| C(28)—C(23)—C(21) | 119.2(4) |
| C(24)—C(23)—C(21) | 120.6(4) |
| C(25)—C(24)—C(23) | 119.4(4) |
| C(24)—C(25)—C(26) | 120.2(4) |
| C(25)—C(26)—C(27) | 120.3(4) |
| C(25)—C(26)—C(29) | 120.0(4) |
| C(27)—C(26)—C(29) | 119.7(4) |
| C(28)—C(27)—C(26) | 118.7(4) |
| C(27)—C(28)—C(23) | 121.3(4) |
| N(30)—C(29)—C(26) | 176.7(5) |
| N(32)—C(31)—C(36) | 123.7(4) |
| N(32)—C(31)—N(20) | 117.0(4) |
| C(36)—C(31)—N(20) | 119.3(4) |
| C(31)—N(32)—C(33) | 116.9(4) |
| N(32)—C(33)—C(34) | 123.7(4) |
| C(33)—C(34)—C(35) | 117.5(4) |
| C(36)—C(35)—C(34) | 120.1(4) |
| C(31)—C(36)—C(35) | 118.1(4) |

TABLE J
Anisotropic displacement parameters (Å² × 10³).
The anisotropic displacement factor exponent takes the
form: −2 π² [h² a * ² U₁₁ + . . . + 2 h k a * b * U₁₂].

| | U11 | U22 | U33 | U23 | U13 | U12 |
|---|---|---|---|---|---|---|
| C(1) | 31(2) | 35(2) | 36(3) | 4(2) | −2(2) | −5(2) |
| C(2) | 36(2) | 33(2) | 34(3) | 0(2) | −1(2) | −1(2) |
| C(3) | 34(3) | 84(3) | 49(3) | 9(3) | 0(2) | 0(2) |
| N(4) | 25(2) | 34(2) | 35(2) | 0(2) | 2(2) | −2(2) |
| C(5) | 40(3) | 32(2) | 39(3) | 0(2) | −3(2) | −1(2) |
| C(6) | 47(3) | 32(2) | 41(3) | −1(2) | −4(2) | −3(2) |
| N(7) | 42(2) | 29(2) | 29(2) | 2(2) | −3(2) | −3(2) |
| C(8) | 39(3) | 32(2) | 37(3) | −1(2) | 1(2) | 1(2) |
| C(9) | 44(3) | 23(2) | 48(3) | 5(2) | −1(2) | −4(2) |
| C(10) | 33(2) | 33(2) | 33(3) | 4(2) | 3(2) | −7(2) |
| C(11) | 30(2) | 27(2) | 39(3) | 4(2) | 3(2) | 2(2) |
| O(12) | 41(2) | 44(2) | 40(2) | 0(2) | 0(2) | 10(1) |
| C(13) | 45(3) | 43(3) | 51(3) | −1(2) | 13(3) | 8(2) |
| C(14) | 62(3) | 37(2) | 49(3) | −2(2) | 0(3) | 11(2) |
| O(15) | 58(2) | 46(2) | 39(2) | −1(2) | 2(2) | 0(2) |
| C(16) | 37(2) | 33(3) | 33(3) | 0(2) | 1(2) | −7(2) |
| C(17) | 42(3) | 37(2) | 46(3) | 6(2) | −14(2) | −5(2) |
| C(18) | 36(3) | 34(2) | 53(3) | 9(2) | −7(2) | 2(2) |
| C(19) | 33(2) | 29(2) | 46(3) | 6(2) | −1(2) | 1(2) |
| N(20) | 39(2) | 30(2) | 32(2) | −5(2) | −2(2) | 0(2) |
| C(21) | 32(2) | 41(3) | 39(3) | 3(2) | 9(2) | 0(2) |
| O(22) | 39(2) | 35(2) | 55(2) | −11(2) | −1(2) | 1(1) |
| C(23) | 25(2) | 32(2) | 37(3) | −6(2) | −1(2) | 7(2) |
| C(24) | 35(3) | 40(2) | 39(3) | 8(2) | 3(2) | −2(2) |
| C(25) | 30(2) | 40(2) | 48(3) | 0(2) | 0(2) | −1(2) |
| C(26) | 35(3) | 34(2) | 33(3) | −3(2) | −4(2) | 9(2) |
| C(27) | 40(3) | 32(2) | 44(3) | 5(2) | −3(2) | 11(2) |
| C(28) | 34(2) | 30(2) | 45(3) | 5(2) | −2(2) | 4(2) |
| C(29) | 52(3) | 27(2) | 55(4) | 3(2) | −7(3) | 10(2) |
| N(30) | 67(3) | 46(2) | 73(3) | −3(2) | −24(3) | 4(2) |
| C(31) | 32(2) | 32(2) | 41(3) | −3(2) | −9(2) | 4(2) |
| N(32) | 55(2) | 31(2) | 51(3) | −4(2) | 3(2) | 0(2) |
| C(33) | 55(3) | 32(3) | 62(4) | 0(3) | −5(3) | 1(2) |
| C(34) | 45(3) | 44(3) | 48(3) | −13(3) | −4(3) | 8(2) |
| C(35) | 46(3) | 55(3) | 50(3) | −1(3) | 14(3) | 2(2) |
| C(36) | 47(3) | 36(2) | 39(3) | −1(2) | 7(2) | 0(2) |
| Cl(37) | 34(1) | 41(1) | 67(1) | 4(1) | 12(1) | 2(1) |

TABLE K
Hydrogen coordinates (× 10⁴) and isotropic displacement parameters
(Å² × 10³).

| U(eq) | x | y | z |
|---|---|---|---|
| H(1) | 8308 | 5687 | 2746 |
| 41 | | | |
| H(2A) | 6685 | 3886 | 3054 |
| 41 | | | |
| H(2B) | 7861 | 2680 | 2884 |
| 41 | | | |
| H(3A) | 10090 | 4962 | 2231 |
| 83 | | | |
| H(3B) | 9924 | 3338 | 2385 |
| 83 | | | |
| H(3C) | 10684 | 4534 | 2671 |
| 83 | | | |
| H(4) | 5910(42) | 4614(34) | 2475(11) |
| 47 | | | |
| H(5A) | 6972 | 2528 | 2176 |
| 45 | | | |
| H(5B) | 8108 | 3455 | 1890 |
| 45 | | | |
| H(6A) | 5855 | 2755 | 1521 |
| 48 | | | |
| H(6B) | 4718 | 3468 | 1851 |
| 48 | | | |
| H(8A) | 4577 | 6000 | 1924 |
| 43 | | | |
| H(8B) | 5607 | 7043 | 1643 |
| 43 | | | |
| H(9A) | 7940 | 6259 | 1957 |
| 46 | | | |
| H(9B) | 6756 | 6892 | 2288 |
| 46 | | | |
| H(13A) | 8702 | 2206 | 607 |
| 56 | | | |
| H(13B) | 8565 | 3834 | 452 |
| 56 | | | |
| H(14A) | 7530 | 2218 | −21 |
| 59 | | | |
| H(14B) | 6270 | 1668 | 304 |
| 59 | | | |
| H(17) | 3596 | 5465 | 73 |
| 50 | | | |
| H(18) | 2527 | 6801 | 600 |
| 49 | | | |
| H(19) | 3409 | 6503 | 1252 |
| 43 | | | |
| H(24) | 11867 | 4848 | 3492 |
| 46 | | | |
| H(25) | 13902 | 5238 | 3953 |
| 47 | | | |
| H(27) | 12366 | 1846 | 4617 |
| 47 | | | |
| H(28) | 10437 | 1401 | 4135 |
| 44 | | | |
| H(33) | 9058 | 8525 | 3505 |
| 60 | | | |
| H(34) | 7951 | 8468 | 4141 |
| 55 | | | |
| H(35) | 7190 | 6236 | 4406 |
| 61 | | | |
| H(36) | 7590 | 4142 | 4029 |
| 49 | | | |

TABLE L
Torsion angles [deg].

| | |
|---|---|
| N(4)—C(1)—C(2)—N(20) | −167.7(3) |
| C(3)—C(1)—C(2)—N(20) | 65.3(4) |
| C(3)—C(1)—N(4)—C(9) | −82.2(4) |
| C(2)—C(1)—N(4)—C(9) | 151.3(3) |
| C(3)—C(1)—N(4)—C(5) | 43.9(4) |
| C(2)—C(1)—N(4)—C(5) | −82.6(4) |
| C(9)—N(4)—C(5)—C(6) | −52.4(4) |
| C(1)—N(4)—C(5)—C(6) | −178.8(3) |
| N(4)—C(5)—C(6)—N(7) | 57.1(4) |
| C(5)—C(6)—N(7)—C(10) | 157.8(3) |
| C(5)—C(6)—N(7)—C(8) | −63.2(4) |
| C(10)—N(7)—C(8)—C(9) | −157.6(3) |
| C(6)—N(7)—C(8)—C(9) | 63.5(4) |
| C(5)—N(4)—C(9)—C(8) | 53.9(4) |
| C(1)—N(4)—C(9)—C(8) | −178.3(3) |
| N(7)—C(8)—C(9)—N(4) | −58.7(4) |
| C(8)—N(7)—C(10)—C(11) | 157.1(3) |
| C(6)—N(7)—C(10)—C(11) | −67.2(5) |
| C(8)—N(7)—C(10)—C(19) | −18.5(5) |
| C(6)—N(7)—C(10)—C(19) | 117.3(4) |
| N(7)—C(10)—C(11)—C(16) | −175.4(3) |
| C(19)—C(10)—C(11)—C(16) | 0.4(5) |
| N(7)—C(10)—C(11)—O(12) | 2.2(5) |
| C(19)—C(10)—C(11)—O(12) | 178.1(3) |
| C(16)—C(11)—O(12)—C(13) | 17.2(5) |
| C(10)—C(11)—O(12)—C(13) | −160.5(3) |
| C(11)—O(12)—C(13)—C(14) | −47.5(4) |
| O(12)—C(13)—C(14)—O(15) | 62.0(5) |
| C(13)—C(14)—O(15)—C(16) | −42.2(5) |
| C(14)—O(15)—C(16)—C(11) | 12.1(5) |
| C(14)—O(15)—C(16)—C(17) | −170.1(3) |
| O(12)—C(11)—C(16)—O(15) | 1.3(6) |
| C(10)—C(11)—C(16)—O(15) | 178.9(3) |
| O(12)—C(11)—C(16)—C(17) | −176.3(3) |
| C(10)—C(11)—C(16)—C(17) | 1.2(6) |
| O(15)—C(16)—C(17)—C(18) | −178.9(3) |
| C(11)—C(16)—C(17)—C(18) | −1.1(6) |
| C(16)—C(17)—C(18)—C(19) | −0.7(6) |
| C(17)—C(18)—C(19)—C(10) | 2.3(6) |
| N(7)—C(10)—C(19)—C(18) | 173.5(3) |
| C(11)—C(10)—C(19)—C(18) | −2.2(5) |
| C(1)—C(2)—N(20)—C(21) | −122.3(4) |
| C(1)—C(2)—N(20)—C(31) | 75.4(4) |
| C(31)—N(20)—C(21)—O(22) | 153.9(4) |
| C(2)—N(20)—C(21)—O(22) | −7.8(6) |
| C(31)—N(20)—C(21)—C(23) | −28.4(5) |
| C(2)—N(20)—C(21)—C(23) | 169.9(3) |
| O(22)—C(21)—C(23)—C(28) | −42.9(5) |
| N(20)—C(21)—C(23)—C(28) | 139.3(4) |
| O(22)—C(21)—C(23)—C(24) | 132.2(4) |
| N(20)—C(21)—C(23)—C(24) | −45.6(5) |
| C(28)—C(23)—C(24)—C(25) | −1.1(6) |
| C(21)—C(23)—C(24)—C(25) | −176.2(4) |
| C(23)—C(24)—C(25)—C(26) | −1.3(6) |
| C(24)—C(25)—C(26)—C(27) | 1.9(6) |
| C(24)—C(25)—C(26)—C(29) | −176.1(4) |
| C(25)—C(26)—C(27)—C(28) | 0.0(6) |
| C(29)—C(26)—C(27)—C(28) | 178.0(4) |
| C(26)—C(27)—C(28)—C(23) | −2.5(6) |
| C(24)—C(23)—C(28)—C(27) | 3.1(6) |
| C(21)—C(23)—C(28)—C(27) | 178.2(3) |
| C(21)—N(20)—C(31)—N(32) | 125.0(4) |
| C(2)—N(20)—C(31)—N(32) | −72.9(4) |
| C(21)—N(20)—C(31)—C(36) | −56.6(5) |
| C(2)—N(20)—C(31)—C(36) | 105.5(4) |
| C(36)—C(31)—N(32)—C(33) | 0.0(6) |
| N(20)—C(31)—N(32)—C(33) | 178.3(4) |
| C(31)—N(32)—C(33)—C(34) | 1.2(6) |
| N(32)—C(33)—C(34)—C(35) | −1.4(7) |
| C(33)—C(34)—C(35)—C(36) | 0.3(6) |
| N(32)—C(31)—C(36)—C(35) | −1.0(6) |
| N(20)—C(31)—C(36)—C(35) | −179.2(3) |
| C(34)—C(35)—C(36)—C(31) | 0.8(6) |

### Example 8

### Amorphous Form

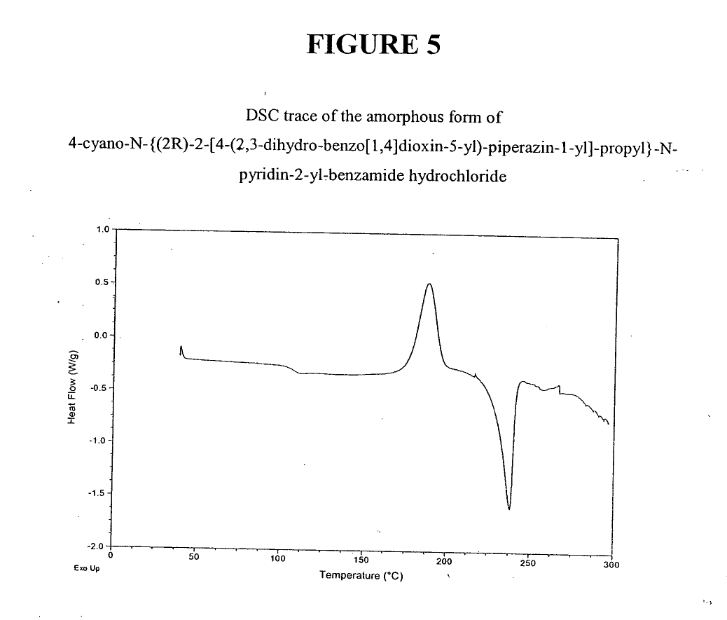

The amorphous form of 4-cyano-N-{(2R)-2-[4-(2,3-dihydro-benzo[1,4]dioxin-5-yl)-piperazin-1-yl]-propyl}-N-pyridin-2-yl-benzamide hydrochloride was prepared by adding abot 100 mg of 4-cyano-N-{(2R)-2-[4-(2,3-dihydro-benzo[1,4]dioxin-5-yl)-piperazin-1-yl]-propyl}-N-pyridin-2-yl-benzamide hydrochloride to 1 ml of water in a vial. The suspension was heated to about 40° C. and stirred until all solids were dissolved. The solution was placed in an oven set to 50° C., and the pressure was gradually reduced to −20 inch of Hg. After 24 hours, the vial was removed, yielding the amorphous material. The X-ray powder diffraction pattern of the amorphous form is shown in FIG. 4. As can be seen, the DSC is substantially devoid of any prominent peaks (reflections). FIG. 5 depicts a differential scanning calorimetry (DSC) trace of the amorphous form.

Other Crystal Forms

A screen was performed to determine the existence of additional crystal forms of 4-cyano-N-{(2R)-2-[4-(2,3-dihydro-benzo[1,4]dioxin-5-yl)-piperazin-1-yl]-propyl}-N-pyridin-2-yl-benzamide hydrochloride.

Reslurry Experiments:

4-cyano-N-{(2R)-2-[4-(2,3-dihydro-benzo[1,4]dioxin-5-yl)-piperazin-1-yl]-propyl}-N-pyridin-2-yl-benzamide hydrochloride was reslurried in about 1-2 ml of ethanol, IPA, ethyl acetate, acetone, THF, acetonitrile, toluene, iso-propyl acetate, and water. The suspensions were stirred in 5 ml vials for 12 days at RT. The suspension samples were withdrawn at days 6 and 12, filtered, and analyzed via XRD while wet. No transformation was noticed, all XRD scans showed Form A (see Table M).

TABLE M
XRD on reslurried samples

| | XRD | XRD |
|---|---|---|
| Solvent | analysis, day 6 | analysis, day 12 |
| Ethanol | A | A |
| IPA | A | A |
| EthOAC | A | A |
| Acetone | A | Not performed |
| THF | A | A |
| Toluene | A | A |
| Iso-propyl acetate | Not performed | A |
| methanol | A | Not performed |
| methanol | A | Not performed |
| water | A | Not performed |
| water | A | Not performed |

Form Screening by Crystallization

Three sets of crystallization experiments were carried out to investigate polymorphism of this compound. In the first set, the compound was recrystallized from conventional solvents using different techniques. In the second set, solids were generated from the reslurry of amorphous material in different solvents including some non-conventional solvents. In the third set, solids were generated through reactive crystallization of HCl and the free base in different solvents.

First Set of Form Screening

4-cyano-N-{(2R)-2-[4-(2,3-dihydro-benzo[1,4]dioxin-5-yl)-piperazin-1-yl]-propyl}-N-pyridin-2-yl-benzamide hydrochloride was crystallized from different solvents by cooling, antisolvent, and evaporative crystallization. Excess API solid was added to different solvents, the suspension stirred for 2 hrs at 60° C., and the undissolved solids were filtered off. About 1.5 ml of each solution was obtained for each experiment.

For fast cooling crystallization, the temperature of the solution was either reduced to room temperature in about 10 minutes, or crash cooled to −15° C. in ice/methanol. Slow cooling was carried out in around 1 hour. Fast antisolvent crystallization was performed by immediately adding several volumes of heptane to the solution at 60° C.; slow addition was carried out in around 30 minutes. In evaporative crystallization, vials containing unsaturated solutions were stirred open-cap for two days (slow evaporation) and placed in an oven at 50° C. and gradually vacuum applied until all solvents evaporated (fast evaporation). Some vials did not generate solids due to very low solubility. In slow cooling with water, the solution oiled out and XRD showed amorphous material. The oiling out in water is associated with the compound's very high solubility in this solvent. Based on XRD scans, Forms A and an amorphous material were generated in these experiments. The results are summarized on Table N.

TABLE N
Screening by Different Crystallization Techniques

| | | | Anti-Solvent | | | |
|---|---|---|---|---|---|---|
| | | | Addition | | | |
| | Cooling | | Heptane | | Evaporative | |
| Solvent | Fast A | Slow B | Fast C | Slow D | Fast E | Slow F |
| Ethanol | A | A | A | A | No solid | |
| IPA | A | No solid | A | No solid | No solid | A |
| EthOAC | No solid | No solid | A | No solid | No solid | |
| Acetone | No solid | No solid | A | No solid | No solid | A |
| THF | No solid | No solid | No solid | No solid | A | A |
| Acetonitrile | A | No solid | No solid | No solid | A | A |
| Toluene | No solid | No solid | No solid | No solid | A | A |
| Isopropyl acetate | No solid | No solid | No solid | No solid | No solid | No solid |
| Water | Oiled out, | No solid | No solid | No solid | No solid | No solid |
| | amorphous | | | | | |

To further investigate the possibility of a hydrate form, saturated solutions of the compound in ethanol, IPA, acetone, THF, and acetonitrile containg 5% water were prepared. The solutions were both crash-cooled and slow-cooled. In these experiments also, only Form A was observed (see Table O).

TABLE O
Results of Cooling Crystallization in Solvents Containing 5% Water

| | XRD analysis, | XRD analysis, |
|---|---|---|
| Solvent | fast cooling in ice: methanol | slow cooling |
| Ethanol: 5% water | A | A |
| IPA: 5% water | A | A |
| Acetone: 5% water | A | A |
| THF: 5% water | A | A |
| Acetonitrile: 5% water | A | A |

Form Screening Starting from Amorphous API

In these experiments, amorphous API was reslurried in different solvents at room temperature. To prepare amorphous API, around 100 mg of the API was added to about 1 ml of water in 22 vials. The suspension heated to about 40° C. and stirred until all solids dissolved. The solutions were placed in an oven and the temperature was set to 50° C.; the pressure was gradually reduced to −20 inch of Hg. After 24 hours, the vials were removed while the solids in the vials were glassy and amorphous. To each vial, between 0.25 to 1 ml of different solvents (see Table P) were added, after 30 minutes to 1 hour stirring at room temperature, some vials contained white suspension indicating potential form transformation. Some vials stirred overnight and the same color change (glassy to white) observed. In some vials, the solids remained glassy, for others the solids fully dissolved due to the high solubility of solids in the solvents. In cases where color change was observed, the suspension was filtered and analyzed by XRD without drying in the oven. According to XRD results, the solids were all Form A (see Table P).

TABLE P
Results of Form Screening with Different Solvents
(starting with amorphous material)

| Solvent | XRD |
|---|---|
| Ethanol | A |
| IPA | A |
| Ethylacetate | A |
| Acetone | A |
| THF | A |
| TBME | A |
| Acetonitrile | A |
| Toluene | A |
| Isopropyl Acetate | A |
| Methanol | dissolved |
| Water | dissolved |
| DMF | dissolved |
| Ethylene Glycol | dissolved |
| t-Butanol | A |
| Dioxane | A |
| Butylacetate | A |
| Di-ethoxy methane | A |
| 3-Pentanone | A |
| 1,2 dimethoxy ethane | A |
| Monochlorobenzene | dissolved |
| 1-Methoxycyclohexane | dissolved |
| Methylsulfoxide | A |

Form Screening by Salt Formation Via Reactive Crystallization

Salt was produced from the free base and HCl solution in both pure and mixed solvents.

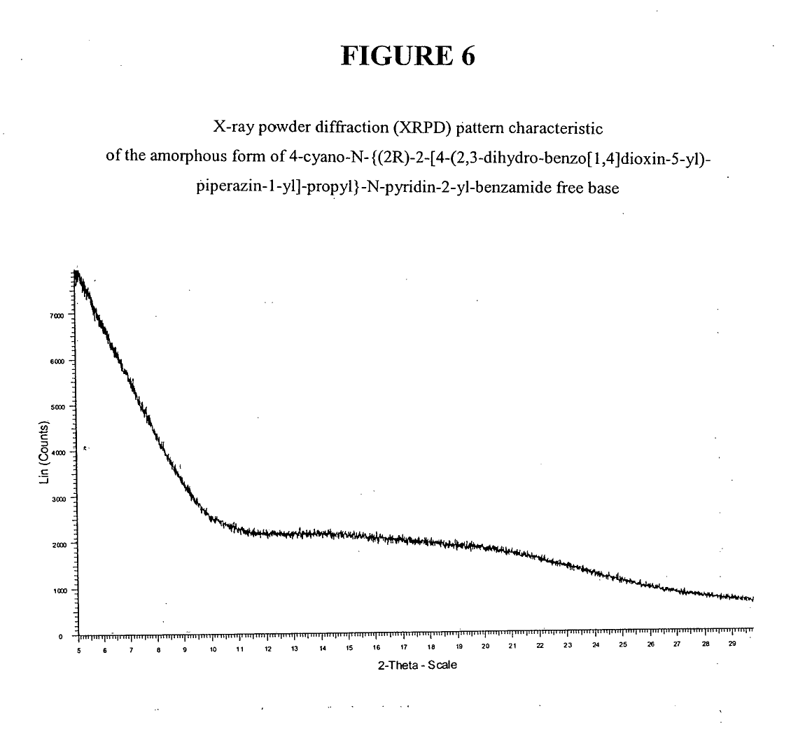

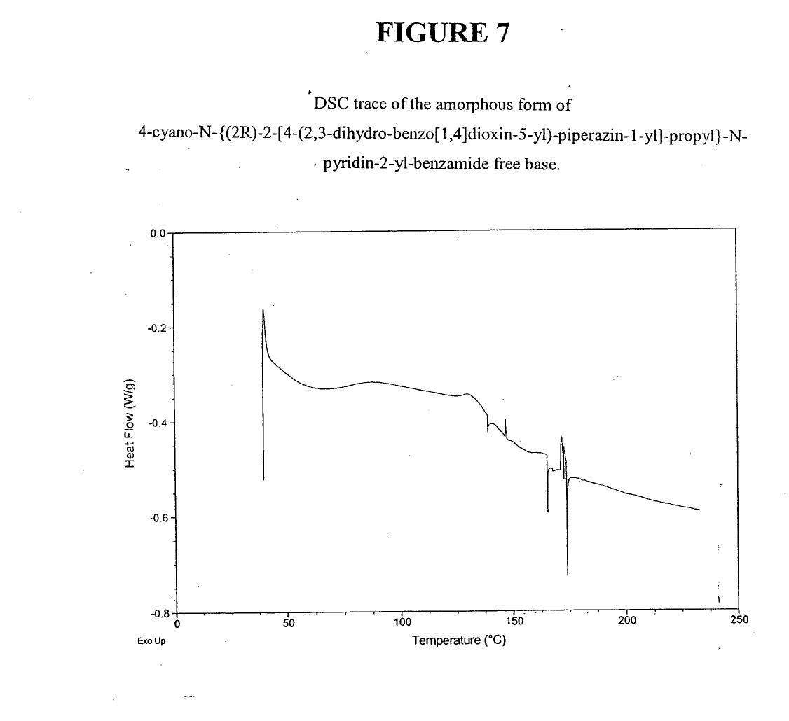

Salt formation in pure solvents: HCl solutions in ethanol, ethyl acetate, t-BME, IPA, and methanol were made. See Table Q for the concentration of HCl in each solvent. Amorphous free base was made by evaporation of the solvent from a free base-ethanol solution. Evaporation was performed by placing the solution in an oven at room temperature under full vacuum for three days. FIGS. 6 and 7 show depict XRD and DSC scans, respectively, of the amorphous free base. 100 mg of the free base and an equivalent mole of the HCl solution was used. In all experiments, 0.25 ml of the solvent was utilized; all experiments were performed at room temperature. The results are shown in Table Q. All experiments except in methanol generated solids of Form A. Crystallization in methanol did not generate solids; evaporation of the solution yielded oily material.

TABLE Q
Form Screening by Salt Formation From Pure Solvents

| | | | Vol. Of | |
|---|---|---|---|---|
| | | Wt of HCl | solvent to | |
| | HCl Conc % | solution | the freebase | Form |
| Solvent | (wt/wt of solv) | (mg) | mg | (XRD) |
| Ethanol | 7.5 | 111 | 0.25 | A |
| Ethyl acetate | 7.3 | 115 | 0.25 | A |
| t-BME | 11% | 76 | 0.25 | A |
| IPA | 15 | 56 | 0.25 | A |
| Methanol | 29 | 29 | 0.25 | — |

Salt generation in mixed solvents: In these experiments salt was generated from free base dissolved in different solvents (see Table R) and HCl solution in either methanol or IPA. The concentration of HCl in methanol and IPA were 15% and 29%, respectively. Results are shown in Table R. No new form was generated in the experiments. Table S shows the onset temperature of thermal events of Form A and amorphous salt.

TABLE R
Form Screening by Salt Formation From Mixed Solvents

| | Solvent in which | |
|---|---|---|
| Solvent | HCl sol. was made | XRD |
| Acetonitrile | Methanol | A |
| DMF | Methanol | A |
| IsoPropyl Acetate | Methanol | A |
| Toluene | Methanol | A |
| THF | Methanol | A |
| Acetone | Methanol | A |
| Mono-Chloro Benzene | Methanol | A |
| Di-Oxane | Methanol | A |
| Butyl Acetate | Methanol | A |
| Cyclo Hexane | IPA | Solution |
| | | degradation |
| 1,2 dimethoxy Ethane | IPA | A |
| Di-etho mathane | IPA | A |
| Methyl Sulfoxide | Methanol | A |
| Ethylene Glycol | Methanol | No solid |
| t-Butanol | Methanol | pasty, |

TABLE S
Thermal Events of Form A and amorphous 4-cyano-N-{(2R)-2-[4-
(2,3-dihydrobenzo[1,4]dioxin-5-yl)-piperazin-1-yl]-propyl}-N-
pyridin-2-yl-benzamidehydrochloride

| Form | Glass transition | Crystallization temp. | Melting Point |
|---|---|---|---|
| A | — | — | 237° C. |
| Amorphous | 111° C. | 188 | 229 |

Polymorph Search by Thermal Operation

Since the amorphous salt undergoes a glass transition, crystallization, and melting events, the existence of a possible new form after crystallization and before melting was investigated. Accordingly, a sample was heated to 188° C. and then cooled and analyzed by XRD. The analysis showed that the amorphous had converted to Form A before melting.

Acquisition of Analytical Data

Differential scanning calorimetry data were collected using a DSC (TA instrument, model Q1000) under the following parameters: 50 mL/min purge gas(N₂); scan range 37 to 300° C., scan rate 10° C./min. X-Ray data was acquired using an X-ray powder diffractometer (Bruker-axs, model D8 advance) having the following parameters: voltage 40 kV, current 40.0 mA, scan range (2θ) 5 to 300, total scan time 20 minutes, with Ni filter, Vantec-1 detector, 1 mm divergence slit.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

What is claimed is:

1. A crystal form of 4-cyano-N-{(2R)-2-[4-(2,3-dihydro-benzo[1,4]dioxin-5-yl)-piperazin-1-yl]-propyl}-N-pyridin-2-yl-benzamide hydrochloride having an X-ray powder diffraction pattern comprising characteristic peaks, in terms of 20, at about 16.8° and about 21.8°; wherein said crystal form is substantially free of hydrocarbon solvents.

2. The crystal form of claim 1 having an X-ray powder diffraction pattern comprising characteristic peaks, in terms of 20, at about 14.3°, about 16.8°, about 21.8°, and about 22.3°.

3. The crystal form of claim 1 having an X-ray powder diffraction pattern comprising characteristic peaks, in terms of 20, at about 14.3°, about 16.14°, about 16.8°, about 19.0°, about 21.8°, and about 22.3°.

4. The crystal form of claim 1 having an X-ray powder diffraction pattern comprising at least 3 characteristic peaks, in terms of 20, selected from about 5.3°, about 10.6°, about 11.6°, about 12.3°, about 14.3°, about 15.0°, about 16.14°, about 16.8°, about 19.0°, about 21.8°, about 22.3°, and about 23.4°.

5. The crystal form of claim 1 having an X-ray powder diffraction pattern substantially as shown in FIG. 1.

6. The crystal form of claim 1 having a differential scanning calorimetry trace showing an endotherm maximum at about 225 to about 245° C.

7. The crystal form of claim 1 having a differential scanning calorimetry trace showing an endotherm maximum at about 230 to about 240° C.

8. The crystal form of claim 1 having a differential scanning calorimetry trace showing an endotherm maximum at about 234° C.

9. The crystal form of claim 1 having a differential scanning calorimetry trace substantially as shown in FIG. 2.

10. The crystal form of claim 1 having a thermogravimetric analysis trace showing about 2.5 to about 7.5% weight loss from about 130 to about 250° C.

11. The crystal form of claim 1 having a thermogravimetric analysis trace showing about 3.5 to about 6.5% weight loss from about 130 to about 250° C.

12. The crystal form of claim 1 having a thermogravimetric analysis trace showing about 4.0 to about 6.0% weight loss from about 140 to about 240° C.

13. The crystal form of claim 1 having a thermogravimetric analysis trace substantially as shown in FIG. 2.

14. A composition comprising the crystal form of claim 1.

15. The composition of claim 14 wherein at least about 50% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

16. The composition of claim 14 wherein at least about 70% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

17. The composition of claim 14 wherein at least about 80% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

18. The composition of claim 14 wherein at least about 90% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

19. The composition of claim 14 wherein at least about 95% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

20. The composition of claim 14 wherein at least about 97% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

21. The composition of claim 14 wherein at least about 98% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

22. The composition of claim 14 wherein at least about 99% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

23. A pharmaceutical composition comprising the crystal form of claim 1 and a pharmaceutically acceptable carrier.

24. A process of preparing a crystal form of 4-cyano-N-{(2R)-2-[4-(2,3-dihydro-benzo[1,4]dioxin-5-yl)-piperazin-1-yl]-propyl}-N-pyridin-2-yl-benzamide hydrochloride, said crystal form having an X-ray powder diffraction pattern comprising characteristic peaks, in terms of 20, at about 16.8° and about 21.8°; said process comprising precipitating the crystal form from a solution of 4-cyano-N-{(2R)-2-[4-(2,3-dihydro-benzo[1,4]dioxin-5-yl)-piperazin-1-yl]-propyl}-N-pyridin-2-yl-benzamide hydrochloride in a crystallizing solvent.

25. The process of claim 24 wherein said solvent comprises an alcohol.

26. The process of claim 24 wherein said solvent comprises ethanol.

27. The process of claim 24 wherein said solvent consists essentially of ethanol.

28. The process of claim 24 wherein said precipitating is carried out by cooling or evaporating said solution.

29. The process of claim 24 wherein said solvent comprises ethanol and said solution is cooled from a temperature of about 50 to about 80° C. to a temperature of about 20 to about −20° C.

30. The process of claim 24 wherein said precipitating is carried out by vapor diffusion.

31. A crystal form prepared by the process of claim 1.

32. A crystal form of 4-cyano-N-{(2R)-2-[4-(2,3-dihydro-benzo[1,4]dioxin-5-yl)-piperazin-1-yl]-propyl}-N-pyridin-2-yl-benzamide hydrochloride having unit cell dimensions a=8.45 Å; b=9.30 Å; c=33.30 Å; and α, β, γ=90°; wherein said crystal form is substantially free of hydrocarbon solvents.

33. The crystal form of claim 32 having orthorhombic space group 2(1)2(1)2(1).

34. The crystal form of claim 33 having atomic coordinates according to Table H.

35. A composition comprising the crystal form of claim 1.

36. The composition of claim 35 wherein at least about 50% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

37. The composition of claim 35 wherein at least about 70% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

38. The composition of claim 35 wherein at least about 80% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

39. The composition of claim 35 wherein at least about 90% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

40. The composition of claim 35 wherein at least about 95% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

41. The composition of claim 35 wherein at least about 97% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

42. The composition of claim 35 wherein at least about 98% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

43. The composition of claim 35 wherein at least about 99% by weight of total 4-cyano-N-{(2R)-2-[4-(2,3-dihydro-benzo[1,4]dioxin-5-yl)-piperazin-1-yl]-propyl}-N-pyridin-2-yl-benzamide hydrochloride in said composition is present as said crystal form.

44. A pharmaceutical composition comprising the crystal form of claim 1 and a pharmaceutically acceptable carrier.

45. A process of preparing a crystal form of 4-cyano-N-{(2R)-2-[4-(2,3-dihydro-benzo[1,4]dioxin-5-yl)-piperazin-1-yl]-propyl}-N-pyridin-2-yl-benzamide hydrochloride, said form having unit cell dimensions a=8.45 Å; b=9.30 Å; c=33.30 Å; and α, β, γ=90°; said process comprising precipitating said crystal form from a solution of 4-cyano-N-{(2R)-2-[4-(2,3-dihydro-benzo[1,4]dioxin-5-yl)-piperazin-1-yl]-propyl}-N-pyridin-2-yl-benzamide hydrochloride in a crystallizing solvent by addition of antisolvent.

46. The process of claim 45 wherein said precipitating is carried out by vapor diffusion.

47. The process of claim 45 wherein said crystallizing solvent comprises ethanol.

48. The process of claim 45 wherein said antisolvent comprises hexanes.

49. A crystal form prepared by the method of claim 45.

50. A method of antagonizing a 5-HT_{1A} receptor comprising contacting the crystal form of claim 1 with said receptor.

51. A method of treating a CNS disorder comprising administering a therapeutically effective amount of a crystal form of claim 1 to a patient in need of said treatment.

52. The method of claim 51 wherein said CNS disorder is schizophrenia, Parkinson's disease, anxiety, Tourette's syndrome, migraine, autism, an attention deficit disorder, a hyperactivity disorder, a sleep disorder, a social phobias, pain, a thermoregulatory disorder, an endocrine disorder, urinary incontinence, vasospasm, stroke, an eating disorders, sexual dysfunction, or alcohol, drug and nicotine withdrawal.

53. A method of treating cognitive dysfunction comprising administering a therapeutically effective amount of a crystal form of claim 1 to a patient in need of said treatment.

54. The method of claim 53 wherein said cognitive dysfunction is associated with mild cognitive impairment (MCI), Alzheimer's disease, Lewy Body dementia, vascular dementia, post stroke dementia, a surgical procedure, traumatic brain injury, or stroke.

55. A method of antagonizing a 5-HT_{1A} receptor comprising contacting a crystal form of 4-cyano-N-{(2R)-2-[4-(2,3-dihydro-benzo[1,4]dioxin-5-yl)-piperazin-1-yl]-propyl}-N-pyridin-2-yl-benzamide hydrochloride having an X-ray powder diffraction pattern comprising characteristic peaks, in terms of 20, at about 16.8° and about 21.8°, with said receptor.

56. A method of treating a CNS disorder comprising administering a therapeutically effective amount of a crystal form of 4-cyano-N-{(2R)-2-[4-(2,3-dihydro-benzo[1,4]dioxin-5-yl)-piperazin-1-yl]-propyl}-N-pyridin-2-yl-benzamide hydrochloride, said crystal form having an X-ray powder diffraction pattern comprising characteristic peaks, in terms of 20, at about 16.8° and about 21.8°, to a patient in need of said treatment.

57. The method of claim 56 wherein said CNS disorder is schizophrenia, Parkinson's disease, anxiety, Tourette's syndrome, migraine, autism, an attention deficit disorder, a hyperactivity disorder, a sleep disorder, a social phobias, pain, a thermoregulatory disorder, an endocrine disorder, urinary incontinence, vasospasm, stroke, an eating disorders, sexual dysfunction, or alcohol, drug and nicotine withdrawal.

58. A method of treating cognitive dysfunction comprising administering a therapeutically effective amount of a crystal form of 4-cyano-N-{(2R)-2-[4-(2,3-dihydro-benzo[1,4]dioxin-5-yl)-piperazin-1-yl]-propyl}-N-pyridin-2-yl-benzamide hydrochloride, said crystal form having an X-ray powder diffraction pattern comprising characteristic peaks, in terms of 2θ, at about 16.8° and about 21.8°, to a patient in need of said treatment.

59. The method of claim 58 wherein said cognitive dysfunction is associated with mild cognitive impairment (MCI), Alzheimer's disease, Lewy Body dementia, vascular dementia, post stroke dementia, a surgical procedure, traumatic brain injury, or stroke.

60. An amorphous form of 4-cyano-N-{(2R)-2-[4-(2,3-dihydro-benzo[1,4]dioxin-5-yl)-piperazin-1-yl]-propyl}-N-pyridin-2-yl-benzamide hydrochloride having an X-ray powder diffraction pattern substantially devoid of characteristic peaks, in terms of 2θ.

61. The amorphous form of claim 60 having an X-ray powder diffraction pattern substantially as shown in FIG. 4.

62. The amorphous form of claim 60 having a differential scanning calorimetry trace showing an endotherm maximum at about 185 to about 190° C.

63. The amorphous form of claim 60 having a differential scanning calorimetry trace showing an endotherm maximum at about 230 to about 240° C.

64. The crystal form of claim 1 that is substantially free of the amorphous form of 4-cyano-N-{(2R)-2-[4-(2,3-dihydro-benzo [1,4]dioxin-5-yl)-piperazin-1-yl]-propyl}-N-pyridin-2-yl-benzamide hydrochloride.
